(19) United States
(12) Patent Application Publication
(10) Pub. No.: US 2014/0228768 A1
(43) Pub. Date: Aug. 14, 2014

(54) HANDHELD MEDICAMENT INJECTION DEVICE WITH ILLUMINATED DOSE BUTTON

(71) Applicant: SANOFI-AVENTIS DEUTSCHLAND GMBH, Frankfurt am Main (DE)

(72) Inventors: Ilona Eggert, Frankfurt am Main (DE); Stuart King, West Midlands (GB)

(73) Assignee: Sanofi-Aventis Deutschland GmbH, Frankfurt am Main (DE)

(21) Appl. No.: 14/346,996

(22) PCT Filed: May 24, 2012
(86) PCT No.: PCT/EP2012/059757

§ 371 Date: Mar. 25, 2014

(30) Foreign Application Priority Data
May 25, 2011 (EP) 11167538.5

(51) Int. Cl. A61M 5/315

(52) U.S. Cl. CPC A61M 5/31535; A61M 2005/3126
USPC 604/189

(57) ABSTRACT

The present invention discloses a handheld medical device having a housing, at least one user activatable button mounted on a surface of the housing, and a light source mounted within the housing below the button and arranged to direct light towards the button. Substantially the whole of the button and the surface of the housing adjacent the button are opaque, save for a narrow strip adjacent the periphery of the button which is non-opaque.

### CROSS REFERENCE TO RELATED APPLICATIONS

The present application is a U.S. National Phase Application pursuant to 35 U.S.C. §371 of International Application No. PCT/EP2012/059757 filed May 24, 2012, which claims priority to European Patent Application No. 11167538.5 filed May 25, 2011. The entire disclosure contents of these applications are herewith incorporated by reference into the present application.

### TECHNICAL FIELD

The present invention relates to a handheld medical device having buttons with novel illumination, particularly but not exclusively a handheld medicament delivery device.

### BACKGROUND

There are a number of handheld medical devices used unsupervised by out-patients. These include, for example, monitoring devices (such as blood glucose monitors) and drug delivery devices. Such devices will generally have a graphical display and a number of buttons with which the user can program or otherwise interact with the device. Providing illuminated buttons to indicate the device being in particular states is known as is the use of flashing buttons. For example, the EasyPod™ injector device has an illuminated dose button that is solid green to indicate an injection is ready, flashing green to indicate an injection is in progress and solid red to indicate an error state.

The use of illuminated buttons is problematic though: aesthetically, achieving even illumination may be difficult. Illuminating a large area may require multiple light sources and increases the power demand of the device. Moreover, although illumination is generally desirable in such devices, illuminated buttons make discreet use of such devices in public difficult. This is an important consideration, particularly for medicament delivery devices, where use of the device in public may be unavoidable but the user may not wish to draw attention to themselves.

The invention was conceived with the above problems in mind.

### SUMMARY

According to a first aspect of the present invention, there is provided a handheld medical device having

a housing,

a user activatable button mounted on a surface of the housing, and

a light source mounted within the housing below the button and arranged to direct light towards the button,

wherein substantially the whole of the button and the surface of the housing adjacent the button are opaque, save for a narrow strip adjacent the periphery of the button which is non-opaque.

It will be understood that in use, light emitted from the light source will emerge from the housing through said narrow strip.

As used herein, “opaque” in respect of a component means that substantially no light passes through it whereas “non-opaque” means that at least some light does pass through, e.g. it is transparent or translucent.

Such illumination is advantageous in that it is aesthetically pleasing, more discreet and potentially lower power demand than total illumination.

Substantially at least 80%, at least 85%, at least 90%, at least 95% or even at least 98% of the button may be opaque. In addition to said narrow strip, it is within the scope of the present invention for the button to include a non-opaque graphic.

It will be understood that there are a number of technical solutions to providing the narrow strip around the periphery of the button (conveniently referred to hereinafter as “edge illumination”).

In certain embodiments edge illumination is achieved by the provision of a non-opaque region in the housing immediately surrounding the button (i.e. the whole of the button save for any graphic is opaque).

In other embodiments edge illumination is achieved by the provision of a clearance between the button and the housing (again the whole of the button save for any graphic is opaque).

In yet further embodiments edge illumination is achieved by the provision of a non-opaque strip extending around the button near its peripheral edge. In certain embodiments the non-opaque strip extend inwardly from the peripheral edge itself.

It will be understood that any of the above methods for producing the edge illumination effect may be used in combination. It will also be understood that in certain embodiments the edge illumination will be provided continuously around the button but that in other embodiments the edge illumination may be discontinuous—either extending only partially around the button or defining a pattern of alternating opaque and non-opaque strips.

More than one light source may be provided, for example to provide illumination of more than one colour (for example any combination of blue, green red or white).

In certain embodiments the light source is one or more LEDs which may be of the same or different colour.

In certain embodiments the device includes a gasket, such as a silicone membrane between the light source and the button to improve sealing of the housing around the button. In such embodiments the gasket is conveniently transparent or translucent. The use of a translucent gasket delivers a soft, even lighting effect.

In particular embodiments the device is a medicament delivery device, in which case the button may be a dose button.

In certain embodiments the device includes a display located in one face of the housing. The button may be in a contiguous face of the housing which face may conveniently be orientated perpendicularly to the face containing the display. Such an arrangement is particularly advantageous when the button is a dose button of a medicament delivery device.

In a variation of the embodiment described in the immediately preceding paragraph, the button may be substantially located in a face contiguous with the face containing the display but also be partially located in the same face as the display. In other words the button is shaped such that it wraps around the interface of the two faces. In such an arrangement edge illumination is provided to at least the edge of the button in the face containing the display. It will be understood that such an arrangement enables the edge illumination to be visible to the user when the device is held in a wider range of positions, for example where the face contiguous with the face containing the button is not visible. This consideration is particularly important when the button is a dose button of a medicament delivery device.

In those embodiments in which the housing incorporates a display, it is particularly convenient for the button illumination to be synchronised with the display. This can be achieved by, for example, providing the device with a programmable microprocessor operatively connected to the display and the light source.

Examples of display/illumination synchronisation include but are not limited to:
flashing button and flashing display (for example to indicate an error condition),
displaying image of button graphic on display while illuminating (perhaps also flashing) that button to convey instructions to user (for example flashing dose button and image of dose button on display (optionally flashing) to indicate dose ready for injection).

The method by which the edge illumination is achieved is not particularly limited. A convenient technique is in mould labelling. A translucent or transparent substrate (e.g. polycarbonate, acrylic or polyester) is printed with an opaque ink save for those regions which are to remain non-opaque in the device. Injection moulding is then effected on the ink side of the substrate (with for example a translucent plastics material). It will be understood that the button can be formed in this way if there is to be a non-opaque graphic and/or edge illumination in the button itself. Alternatively (or in addition) a part of the housing, e.g. a bezel surrounding the button could be formed this way if the edge illumination is in the housing itself.

The term “medicament delivery device” as used herein, means a device capable of administering a dose of one or more medicaments to a patient. Such devices may administer fixed and/or variable doses of medicament to a patient. Handheld medicament delivery devices are sometimes called ‘pen-type’ devices. The medicament delivery mechanism employed by such devices is preferably electromechanical, utilising a motor and gearing to drive a piston rod, although manual delivery mechanisms incorporated into electrically controlled or configured devices may also be envisaged.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compounds,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (−150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopaedia of Pharmaceutical Technology.

Pharmaceutically acceptable solvates are for example hydrates.

### BRIEF DESCRIPTION OF THE FIGURES

The invention will now be further described by way of example with reference to the accompanying drawings, in which:

FIG. 1a is a plan view of a medicament delivery device according to the present invention;

FIG. 1b is a plan view of the device of Fig lb showing illumination of the dose button and the buttons;

FIG. 2 is a perspective view of the dose button in situ in the device of FIG. 1a

FIG. 3 is a simplified cross-sectional view of the dose button assembly of FIG. 1 taken through the plane perpendicular to the front of the device.

References to the device in the following detailed description are intended to refer to the device as referenced in the appended figures and not to when the device is in a use state. Furthermore, the figures are intended to be schematic representations to highlight relevant functionality of the present invention and therefore unnecessary structures have been omitted from the device for clarity. The relative dimensions of the device are also illustratory only. Reference to ‘distal’ and ‘proximal’ are intended to refer to the end of the device where medicament delivery occurs and the opposite end pointing away from the delivery site respectfully.

### DETAILED DESCRIPTION

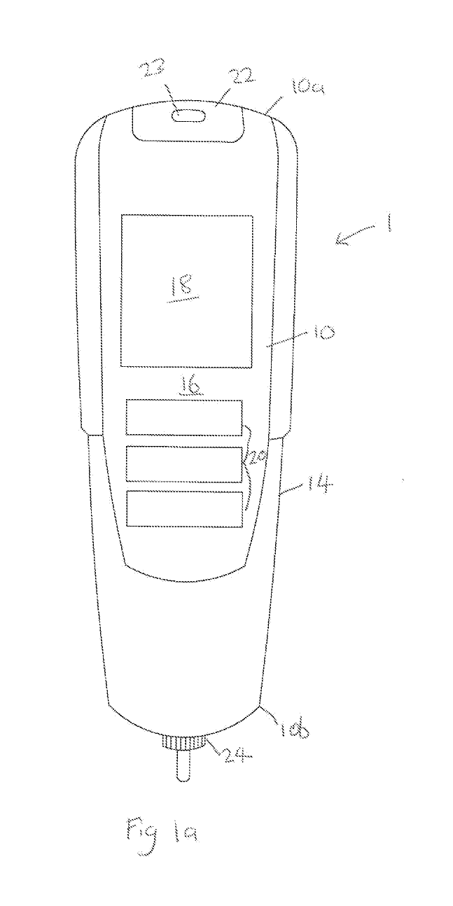

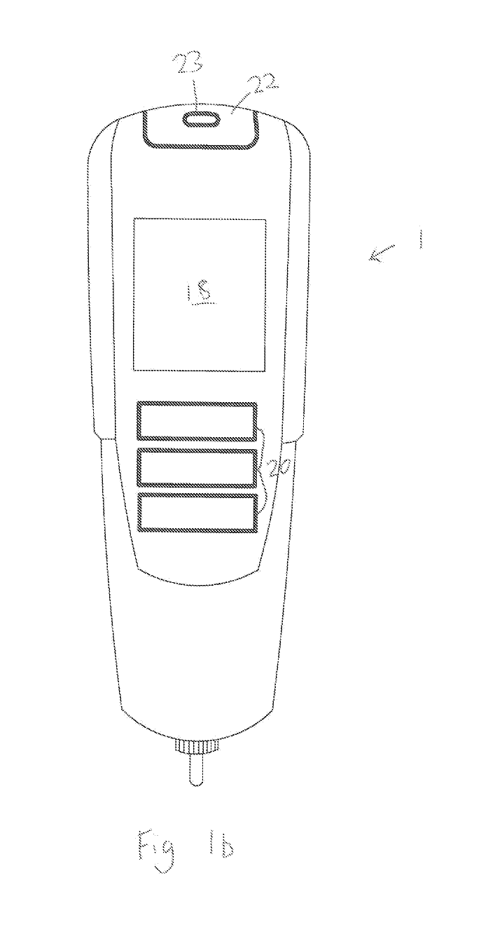

The medicament delivery device 1 illustrated in FIG. 1 comprises a housing 10 having a proximal end 10a and a distal end 10b. At the distal end 10b, the housing is shaped to receive a removable end cap or cover 12 *not shown). This end cap 12 and the housing 10 (at its proximal end) are shaped to provide a form fit connection so that once the cap 12 is slid onto the distal end 10b of the housing 10, the frictional fit between the cap 12 and the housing 10 prevents the cap from inadvertently falling off the housing 10. It will be understood that in other embodiments (not shown) other means of releasably securing the cap to the housing such as snap-fit may be employed.

The interior surface of the cap 12 and the outer surface of the housing 10 at its proximal end 10b are shaped such that there is only one possible configuration in which the cap 12 properly fits onto the distal end 10b of the housing 10. Such an arrangement is preferable because it provides certainty in the alignment of components of the cap 12 with components of the housing 10, as will be explained below.

The housing 10 contains a micro-processor control unit, a printed circuit board (PCB), an electro-mechanical drive train, a battery, and at least one medicament reservoir. A cartridge holder 14 can be removably attached to the housing 10 and may contain one or more cartridges of medicament. The cartridge holder 14 is configured so as allow the replacement of the medicament cartridges as necessary. The medicament delivery device 1 can be used to administer a computed dose of a medicament (or medicaments) through a needle assembly, such as a double ended needle assembly. It will be understood that the cap and housing arrangement described is equally applicable to needleless jet injectors.

A control panel region is provided on one major face 16 of the housing 10 and comprises a digital OLED display 18 towards the distal end 10a of the housing 10 along with a plurality of human interface elements for user input (buttons 20 in the embodiment shown) that can be manipulated by a user, for example to set and inject a medicament dose. It will be understood that in other embodiments (not shown) different display technology such as LCD displays can be used. The buttons 20 also allow navigation through menu structures displayed on the OLED display 18. A dose button 22 (described in more detail below) is provided in a minor (end) face of the housing 10 at its proximal end 10a. In the embodiment shown, the dose button is substantially opaque. For example, the dose button may be substantially black. At the distal end of the housing is provided a screw-threaded needle mount 24. The needle mount 24 is configured to receive a needle hub (not shown). This needle hub can be configured to allow a dose dispenser, such as a conventional pen type injection needle assembly, to be removably mounted to the housing 10. It will be understood that the attachment between the needle mount 24 and a needle hub is preferably a screw fit to allow standard ‘type A’ needles to be fitted to the needle mount 24, although other attachment mechanisms as known in the art, such as Luer lock attachments may be used in other embodiments (not shown).

In use, when the device is turned on, the digital display 18 shown in FIG. 1 illuminates and provides the user certain device information, preferably information relating to the medicaments contained within the cartridge holder 14. For example, the user is provided with certain information relating to both the contents of the cartridge and previous dose history.

FIG. 1b shows a plan view of the device of FIG. 1a, however in FIG. 1b, areas around the dose button 22 and the other device buttons 20 have been illuminated to outline the buttons 20, 22 and improve visibility. Said illumination provides a white or coloured ambient lighting around the opaque keys to emphasise the location and demarcation between the keys. Additionally, further colours can be used to provide status indications to the user. These may include a red glow to indicate a device error state and a blue state upon start up. Furthermore the keys contain additional illuminated graphics, such as the concentric ovals 25 on the dosing button 22 which indicates the preferred location for the user to press the button 22. It can of course be appreciated that embodiments of the invention use alternative arrangements for the illumination and graphics.

The graphics utilised and illuminated on the buttons may also be graphically represented on the OLED screen 18. For example, the ovals 25 and the dose button 22 may be represented on screen to provide a link to the user between the on-screen instructions and the device. Furthermore the light sources may flash in time with the graphic on the screen 18 to further highlight and direct the user. In an example embodiment, also the colour or the colours of the graphics as illuminated by the light sources are represented on the OLED screen 18. In addition, a text representation of the illumination may be shown on the screen. For example, the illuminated dose button is flashing with red light. In a given instance, this may be caused by the user trying to dial a dose after a needle exchange without priming the device (that is expelling a small amount of medicament in order to fill the needle.) At the same time, the display shows a flashing red dose button along with a message “Error: Please prime the device before dialling a dose”.

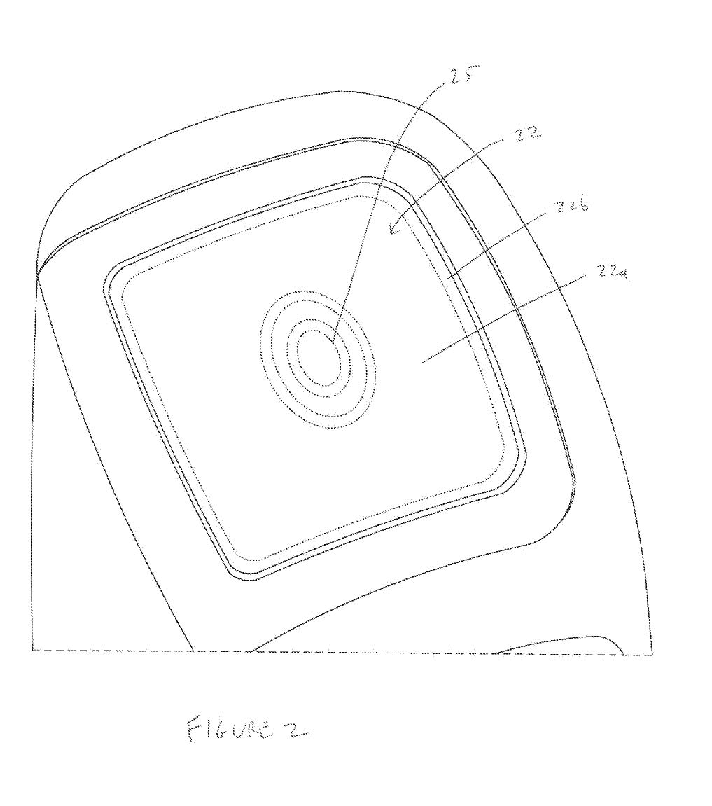

Referring to FIG. 2, the main central area 22a of the button 22 is opaque, whereas a peripheral strip 22b extending around the main central area 22a is non-opaque. This non-opaque strip 22b allows a correspondingly narrow strip of light from an underlying light source to pass out of the device to create the edge illumination effect shown in FIG. 1b. In addition a central logo, in this case concentric ovals 25 is also non-opaque. The combination of opaque and non-opaque regions 22a,22b,25 is conveniently achieved by in mould labelling (IML), a well known moulding technique.

In other embodiments (not shown) edge illumination is achieved by the provision of a clearance between the buttons and the housing 10 (again the whole of the button save for any graphic is opaque).

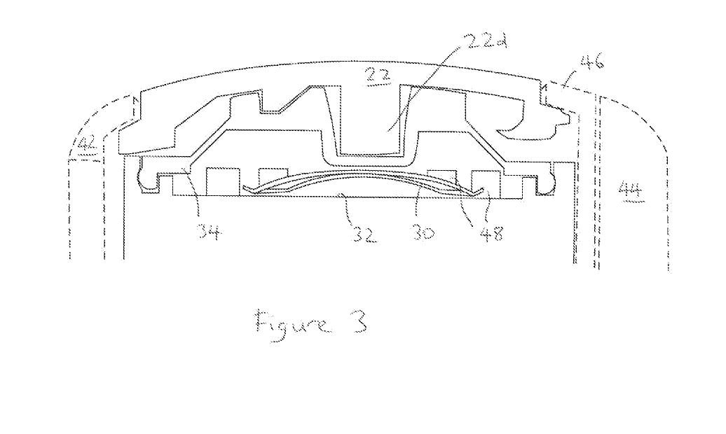

FIG. 3 is a cross section through the centre of the device through the front and rear faces showing the dose button assembly. Essentially the dose button 22 is mounted within a housing bezel 46 within which it is a substantially flush fit, the bezel 46 being retained by front and rear housing parts 42, 44. The button 22 has a downwardly depending peg 22d which acts on a dome switch 30 mounted immediately below the peg 22d on a support surface 32 within the housing 10. Interposed between the button 22 and the dome switch 30 is a flexible silicone gasket 34. Also mounted on the support surface 32 is at least one electronic light source, for example a side firing LEDs 48 (i.e. light is emitted substantially parallel to the button surface). In order to allow light to escape the housing 10 the gasket 34 is translucent to provide a soft diffusion of the light from the LED or LEDs 48. In an embodiment not shown, the dome switch is provided with openings to allow light to pass through the centre of the switch with an LED being located within the dome switch.

In a variation of the embodiment described in the immediately preceding paragraph, the dose button is substantially located in the proximal end face of the device but is also partially located in the same major face as the display. In other words the button is shaped such that it wraps around the interface of the two faces. It will be understood that such an arrangement enables the edge illumination to be visible to the user when the device is held in a wider range of positions, for example where the end face containing the dose button is not itself visible.

1-13. (canceled)

14. A handheld medical device having
a housing,
at least one user activatable button mounted on a surface of the housing, and
a light source mounted within the housing below the button and arranged to direct light towards the button,
wherein substantially the whole of the button and the surface of the housing adjacent the button are opaque, save for a narrow strip adjacent the periphery of the button which is non-opaque,
wherein the button is substantially in a face of the housing contiguous with the face comprising the display, which face is orientated perpendicularly to the face containing the display, and
wherein the button is also partially located in the same face as the display.

15. The device of claim 14, wherein a non-opaque region in the housing immediately surrounding the button is provided.

16. The device of claim 14, wherein a clearance between the button and the housing is provided.

17. The device of claim 14, wherein a non-opaque strip extending around the button near or at its peripheral edge is provided.

18. The device of claim 14, wherein the light source is an LED.

19. The device of claim 14, wherein more than one light source is provided for the button.

20. The device of claim 19, wherein, the light sources are different colours.

21. The device of claim 14, wherein the device includes a transparent or translucent gasket, between the light source(s) and the button to improve sealing of the housing around the button.

22. The device of claim 14, wherein the device is a medicament delivery device.

23. The device of claim 22, in which the button is a dose button.

24. The device of claim 14 additionally comprising a display located in one face of the housing.

25. The device of claim 24 additionally comprising a programmable microprocessor operatively connected to the display and the light source(s) such that button illumination is synchronised with the display.

26. The device of claim 14, wherein at least 80% of the button is opaque.
